(19) United States
(12) Patent Application Publication
(10) Pub. No.: US 2006/0184099 A1
(43) Pub. Date: Aug. 17, 2006

(54) Variable lumen guiding catheter

(76) Inventor: Mun Kyung Hong, New York, NY (US)

Correspondence Address: Browdy & Neimark, PLLC, Suite 300, 624 Ninth Street, N.W., Washington, DC, 20001-5303, US

(21) Appl. No.: 11/293,208

(22) Filed: Dec. 5, 2005

Related U.S. Application Data
(60) Provisional application No. 60/633,574, filed on Dec. 6, 2004.

(51) Int. Cl. A61M 3/00

(52) U.S. Cl. 604/43

(57) ABSTRACT

A method of reducing an amount of contrast material delivered to a patient during an imaging procedure. The method comprising: (a) injecting contrast material through a catheter with a first cross-section; and (b) increasing said cross-section of said lumen to a second cross-section greater than said first cross-section along a portion of the lumen comprising 10% to 90% of a length of said lumen; wherein said increasing occurs after said injection.

### RELATED APPLICATION DATA

The present application claims the benefit under 35 USC 119(e) of U.S. Provisional Application No. 60/633,574 filed on Dec. 6, 2004 by Dr. Mun Hong and entitled “VARIABLE LUMEN GUIDING CATHETER”, the disclosure of which is incorporated herein by reference.

### FIELD OF THE INVENTION

The present invention relates to a medical catheter with a lumen having an adjustable cross-section along at least a portion of its length.

### BACKGROUND OF THE INVENTION

Many medical imaging procedures rely upon delivery of a contrast material through a catheter. The contrast materials are often toxic and problems with contrast material toxicity are serious and widespread. One serious symptom of the toxicity is renal dysfunction.

Currently available measures to reduce the risk of nephropathy from contrast material toxicity include hydration therapy, N-acetylcysteine infusion, and sodium bicarbonate infusion. These measures all require IV administration of fluids.

Renal dysfunction is quite prevalent in patients with coronary and peripheral artery disease, and its worsening status following angioplasty is associated with procedural and later occurring complications. Despite the great advances in guiding catheter technology, no completely satisfactory solution appears to be available.

U.S. Pat. No. 4,406,656 to Hattler teaches a venous catheter having a central flexible lumen and a plurality of collapsible lumens mounted around the periphery of the central flexible lumen.

U.S. Pat. No. 6,013,068 to Spiegelhalter and U.S. Pat. No. 6,733,473 to Reifart et al. each teach catheters with multiple lumens.

U.S. Pat. No. 6,056,719 to Mickley teaches a catheter with a tube near its proximal end to facilitate insertion of an additional guidewire. The tube may be collapsed when not in use.

U.S. Pat. No. 5,002,558 to Klein et al. teaches an adjustable catheter with a dilating balloon length and means to expand and contract the balloon to increase/decrease an external dimension of the catheter.

U.S. Pat. No. 6,776,765 to Soukoup et al. and US patent Application 2005/0010095 by Stewart teach adjustable tensioning mechanisms for adjusting a shape of a catheter.

### SUMMARY OF THE INVENTION

An aspect of some embodiments of the present invention relates to imparting a narrow lumen responsiveness to a wide lumen catheter. In an exemplary embodiment of the invention, the flow resistance properties of an angiography/guiding catheter are imparted to a dual purpose catheter. In an exemplary embodiment of the invention, a 6F (French) dual purpose catheter is perceived by an operator as having injection resistance characteristics of a 3F or 4F catheter. Optionally this causes an operator to inject contrast material with more force. Optionally, the additional force alters flow properties of contrast material exiting a distal tip of the catheter. In an exemplary embodiment of the invention, the altered flow properties permit acquisition of a satisfactory image with a smaller volume of contrast material.

In an exemplary embodiment of the invention, injection resistance properties are altered by reducing a cross-sectional area of a significant portion of a length of the catheter. In another exemplary embodiment of the invention, injection resistance properties are altered by reducing a cross-sectional area of only a small portion. Optionally, reducing a cross-sectional area is accomplished via an internal mechanism in the catheter and/or via a section adapted to be deformed by an external force without undergoing permanent deformation. Optionally, reduction in cross-sectional area may be applied to a portion of the catheter inside a body of a patient and/or outside a body of a patient.

An aspect of some embodiments of the present invention relates to a method of reducing an amount of contrast material delivered to a patient during an imaging procedure. In an exemplary embodiment of the invention, a lumen volume of the catheter is reduced for contrast material injection and increased to facilitate introduction of a medical tool into the body via the catheter lumen.

An aspect of some embodiments of the present invention relates to a cardiac catheter with an adjustable cross-sectional area. Optionally, the cross-sectional area is adjustable over a significant portion of the length of the catheter. Optionally, the cross-sectional area of the catheter is adjustable by, for example from 25% to 50% or lesser or greater or intermediate values. In an exemplary embodiment of the invention, a total volume of contrast material which may reside in the catheter at any given moment is reduced by one fifth, to one half or lesser or greater or intermediate values. In an exemplary embodiment of the invention, a total volume of contrast material which may reside in the catheter defines a residual volume which remains in the catheter after injection.

An aspect of some embodiments of the present invention relates to a mechanism for adjusting a cross-sectional area of a lumen of a cardiac catheter. In an exemplary embodiment of the invention, an adjustable tensioning mechanism operates to collapse a portion of a lumen of the catheter as a means of reducing a cross-sectional area. Optionally, the collapse is non-radical or radial.

In an exemplary embodiment of the invention, a method of reducing an amount of contrast material delivered to a patient during an imaging procedure is provided. The method comprising:
(a) injecting contrast material through a catheter with a first cross-section; and
(b) increasing said cross-section of said lumen to a second cross-section greater than said first cross-section along a portion of the lumen comprising 10% to 90% of a length of said lumen;
   wherein said increasing occurs after said injection.

Optionally, increasing said cross-section of said lumen is applied along a portion of the lumen comprising 15% to 80% of a length of said lumen.

Optionally, increasing said cross-section of said lumen is applied along a portion of the lumen comprising 25% to 75% of a length of said lumen.

Optionally, increasing said cross-section of said lumen is applied along a portion of the lumen comprising 40% to 70% of a length of said lumen.

Optionally, increasing said cross-section of said lumen is applied along a portion of the lumen comprising 50% to 70% of a length of said lumen.

Optionally, said increasing said cross-section of said lumen includes removing an object from said lumen.

Optionally, the method additionally comprising:
(c) providing a catheter with said second cross-section; and
(d) reducing said second cross-section to said first cross-section; and
(e) maintaining said first cross-section during cessation of said injecting.

Optionally, wherein said reducing occurs during said injection.

Optionally, a method additionally comprising conducting an imaging procedure after or during (a).

Optionally, a method additionally comprising inserting a medical tool via said lumen of said catheter after (b).

Optionally, a method additionally comprising performing a procedure with said tool.

In an exemplary embodiment of the invention, there is provided a coronary catheter comprising;
(a) a lumen having a cross-sectional area; and
(b) a collapse mechanism adapted to switch said cross-sectional area between a first configuration and a second configuration over a portion of the catheter, said portion comprising between 10% and 90% of a total catheter length.

Optionally, said portion of the catheter is a contiguous portion.

Optionally, said portion of the catheter is a non-contiguous portion.

Optionally, said portion of the catheter does not include a distal region of the catheter.

Optionally, said distal region of the catheter has a length in the range of 1 to 20 cm measured from a distal tip of the catheter.

Optionally, said collapse mechanism provides a non-radial change in said cross-sectional sectional area.

Optionally, said collapse mechanism is capable of reducing said cross-sectional area by at least 25%.

Optionally, said collapse mechanism is capable of reducing said cross-sectional area by no more than 50%.

Optionally, said switch between said first configuration and said second configuration alters a total volume of said lumen of the catheter by at least 33%.

Optionally, the catheter is supplied as an arterial catheter.

Optionally, the catheter is characterized in that it has sufficient pushability to permit insertion in the face of a resistive force.

Optionally, the catheter includes a lumen which is partially, but not completely, filled with a contrast material.

Optionally, the collapse mechanism comprises:
(a) a series of structures deployed along a length of a wall of a lumen of a catheter, each of said structures attached to said wall; and
(b) a mechanism adapted to move said structures in concert to alter said cross-sectional area of said lumen.

Optionally, said series includes at least two sub series, and a length of wall of said lumen devoid of said structures is interposed between each pair of said at least two sub-series.

Optionally, said structures include arches.

Optionally, said structures are attached to said wall by being embedded therein.

Optionally, said structures are characterized by a height which does not exceed a radius of said lumen.

Optionally, said structures are characterized by a height equal to a radius of said lumen.

In an exemplary embodiment of the invention, there is provided a mechanism for adjusting a cross-sectional area of a lumen of a catheter comprising:
(a) a series of structures deployed along a length of a wall of a lumen of a catheter; and
(b) a mechanism adapted to rotate said structures in concert to alter a cross-sectional area of said lumen.

In an exemplary embodiment of the invention, there is provided a method of reducing an amount of contrast material delivered to a patient during an imaging procedure comprising:
(a) reducing a cross-sectional area of a portion of a lumen of a catheter from an original cross-sectional area to a reduced cross-sectional area;
(b) injecting contrast material; and
(c) increasing said cross-sectional area of said lumen to a cross-sectional area greater than said reduced cross-sectional area.

Optionally, said increasing comprises restoration of said cross-sectional area of said lumen at least to said original cross-sectional area.

In an exemplary embodiment of the invention, there is provided a catheter with an adjustable lumen volume, the catheter comprising a mechanism to reduce a cross-sectional area of at least two separate portions of the catheter, said separate portions characterized by an aggregate length comprising between 10% and 90% of a total catheter length.

Optionally, said mechanism does not reduce a cross-sectional area of at least one intervening portion between said two separate portions.

In an exemplary embodiment of the invention, there is provided a method of reducing an amount of contrast material injected during an imaging procedure conducted with a dual purpose catheter, the method comprising causing an increased resistance to flow of a contrast material.

Optionally, said increased resistance to flow causes an increase in injection pressure.

Optionally, said increased resistance to flow alters flow properties of said contrast material during ejection from a catheter.

In an exemplary embodiment of the invention, there is provided a system for reducing an amount of contrast material injected during an imaging procedure conducted with a dual purpose catheter, the system comprising:
(a) a dual purpose catheter operably connectable to a manual injector;
(b) said manual injector containing a contrast material; and
(c) a regulation mechanism adapted to increase flow resistance to a degree perceivable by an operator of said manual injector.

Optionally, said regulation mechanism operates by altering a cross-sectional area of a portion of a lumen of said catheter.

In an exemplary embodiment of the invention, there is provided a catheter with an adjustable fluid resistance characteristic, the catheter comprising:
(a) a lumen with a cross-sectional area; and
(b) a shielding mechanism to shield a portion of said cross-sectional area from a flow of contrast material over 10 to 90% of a length of said lumen.

Optionally, operation of said mechanism shields at least 40% of said cross-sectional area from said flow of contrast material.

Optionally, operation of said mechanism shields at least 50% of said cross-sectional area from said flow of contrast material.

Optionally, operation of said mechanism shields at least 70% of said cross-sectional area from said flow of contrast material.

In an exemplary embodiment of the invention, there is provided a catheter, said catheter comprising a lumen, said lumen characterized by a cross-sectional area sufficient to permit passage of a tool and contrast material occupying no more than 50% of said cross-sectional area of said lumen over at least 50% of a length of said lumen.

### BRIEF DESCRIPTION OF DRAWINGS

In the Figures, identical structures, elements or parts that appear in more than one Figure are generally labeled with the same numeral in all the Figures in which they appear. Dimensions of components and features shown in the Figures are chosen for convenience and clarity of presentation and are not necessarily shown to scale. The Figures are listed below.

FIG. 1 is a flow diagram illustrating a medical imaging procedure according to some embodiments of the invention:

FIGS. 2A and 2B are side views of a catheter according to an exemplary embodiment of the invention in an uncollapsed state (2A) and a collapsed state (2B);

FIGS. 3A, 3B and 3C are transverse cross-section of a collapsed catheter as depicted in FIG. 2B at lines A-A; B-B and C-C respectively;

FIG. 3D illustrates an arch employed in an exemplary embodiment of a collapse mechanism employed to achieve the collapse of FIG. 2B; and

FIGS. 3E and 3F are side cross-sectional views of a series of arches according to the exemplary embodiment FIG. 3D in an uncollapsed state (3E) and a collapsed state (3F).

### DETAILED DESCRIPTION OF EMBODIMENTS

Overview

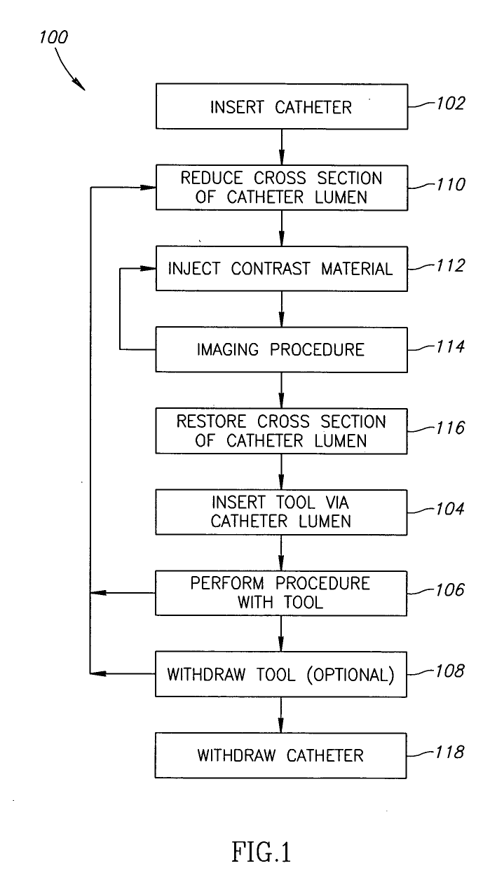

FIG. 1 is a flow diagram illustrating exemplary methods 100 of employing a dual purpose catheter to perform an imaging procedure 114 and a medical procedure 106.

In current medical practice, an imaging procedure is typically conducted with a narrow catheter (e.g. 3F or 4F). Medical procedures such as angioplasty and/or stent delivery typically employ a wider catheter (e.g. 6F) to permit delivery of medical tools through a lumen of the catheter.

In an exemplary embodiment of the invention, handling characteristics of a narrow catheter are imparted to a wider catheter. Optionally, a 6F (French) dual purpose catheter is perceived by an operator as having handling characteristics of a 3F or 4F catheter. This may cause an operator of the catheter to inject contrast material with more force. The additional force optionally alters flow properties of contrast material exiting a distal tip of the catheter. In an exemplary embodiment of the invention, the altered flow properties permit acquisition of a satisfactory image with a small volume of contrast material. Optionally, the handling characteristics are altered by changing a cross-sectional area of at least a portion of a flow path of injected contrast material. Handling characteristics optionally are influenced by previous experience of an operator of the catheter with contrast material injection.

Alternatively or additionally, much of the contrast material remaining in a lumen of a dual purpose catheter when imaging is completed finds its way into the body of the subject. Contrast material remaining in the lumen after imaging is completed may, for example, be pushed into the body by a stent and/or angioplasty balloon delivered through the lumen.

In an exemplary embodiment of the invention, collapse of a portion of a lumen of the catheter before the contrast is introduced into it, reduces a volume of the lumen and a volume of contrast material present in the lumen. Optionally, the reduction in volume of the contrast material in the lumen reduces a volume of contrast delivered into the bloodstream of a patient during insertion of a stent, balloon or other tool. Optionally, the reduction in volume of the contrast material in the lumen alters handling characteristics of the catheter and indirectly contributes to a reduction in the volume of contrast actively injected.

Operational components of catheters according to exemplary embodiments of the invention will be described in detail after the mode of use of a catheter according to the invention is described.

Mode of Use

As illustrated in FIG. 1, a medical procedure 100 typically begins with insertion 102 of a catheter into a body of a subject. In an exemplary embodiment of the invention, the catheter is an arterial catheter. One difference between arterial catheters and venous catheters is the degree of pushability they exhibit. In general, arterial catheters are more pushable than venous catheters. Bends and partial obstructions of arteries can provide a resistive force which the arterial catheter overcomes during insertion. For this reason, arterial catheters are generally more rigid than venous catheters.

In order for a single catheter to permit an imaging procedure 114 and a medical procedure 106, a lumen of the catheter has a diameter sufficiently large to permit introduction of a tool used in performing the procedure. The tool may be, for example, an angioplasty balloon or a stent. Sufficient diameter may be, for example, provided by a 6F catheter.

In some cases, a lumen containing contrast material resides completely within the body of a subject. In other cases, a portion of the lumen resides partly outside a body of the subject. According to various embodiments of the invention, portions of the lumen residing inside and/or outside the body may be affected.

After insertion 102, imaging procedure 114 is conducted. In a typical imaging procedure a subject receives a first aliquot of contrast material which is actively injected 112 for imaging of a target during imaging procedure 114.

The subject also receives a second aliquot of contrast material comprising contrast material remaining in the lumen of the catheter after imaging procedure 114 is concluded. The second aliquot is typically driven into the blood stream by insertion 104 of a medical tool along a lumen of the catheter to perform a medical procedure 106. The medical procedure may be, for example, balloon angioplasty or stent deployment.

According to exemplary embodiments of the invention, a volume of the first aliquot and/or the second aliquot may be reduced.

A method 100 according to the present invention includes reducing 110 a cross-sectional area of at least a portion of a lumen of the catheter. The reduction in the size of the first and/or second aliquot of contrast material achieved by the invention optionally increases as the relative length of catheter lumen subjected to the reduction in cross-sectional area increases and/or as the degree to which the cross-sectional area is decreased increases.

In an exemplary embodiment of the invention, injection 112 of contrast material commences after reduction 110 of cross-sectional area of a portion of the lumen. It is noted that while the catheter is generally to be inserted in its expanded (full size profile) form, it could, under some circumstances be inserted in its collapsed form, especially if injection is to be performed during the process of bringing the catheter to the contrast injection site.

Imaging procedure 114 is then conducted with contrast material flowing outwards from a distal tip of the catheter. Optionally, injection 112 continues and/or is repeated throughout imaging procedure 114. Optionally, reduction 110 of a cross-sectional area of a portion of the lumen causes an operator to increase an applied injection pressure. Optionally, injection pressure is increased to overcome a perceived resistance. Optionally, the increase in applied injection pressure increases a flow rate of contrast material exiting the distal tip.

Possibly, the increased flow rate facilitates improved mixing of the contrast material and reduces a volume required of the first aliquot of contrast material.

When imaging 114 has been completed, the lumen cross-section is restored 116 to its extended dimensions to facilitate insertion 104 of a medical tool (e.g. angioplasty balloon or stent). Restoration of cross-sectional area may cause some blood to enter the catheter. In an exemplary embodiment of the invention, an anti-clotting agent is mixed with the contrast material to prevent clotting of blood in the catheter. Passage of the medical tool along the lumen pushes residual contrast material in the lumen towards a distal tip of the catheter and into the blood stream. In an exemplary embodiment of the invention, the amount of contrast material pushed by the tool is reduced because of the smaller volume of the catheter during the imaging procedure and/or because a portion of the lumen is empty when the tool passes through it.

In an exemplary embodiment of the invention, tool insertion 104 and performance of a medical procedure 106 occur after imaging. When the imaging 114 and medical procedure 106 have been performed, the catheter is withdrawn 118.

In an exemplary embodiment of the invention, after a medical procedure 106 is completed, the sequence of collapse 110, injection 112 and imaging 114 are repeated. Optionally, tool withdrawal 108 occurs before or after this repetition. In exemplary embodiments of the invention in which the tool is a stent, withdrawal 108 optionally is not performed.

Physical Configuration of Catheter

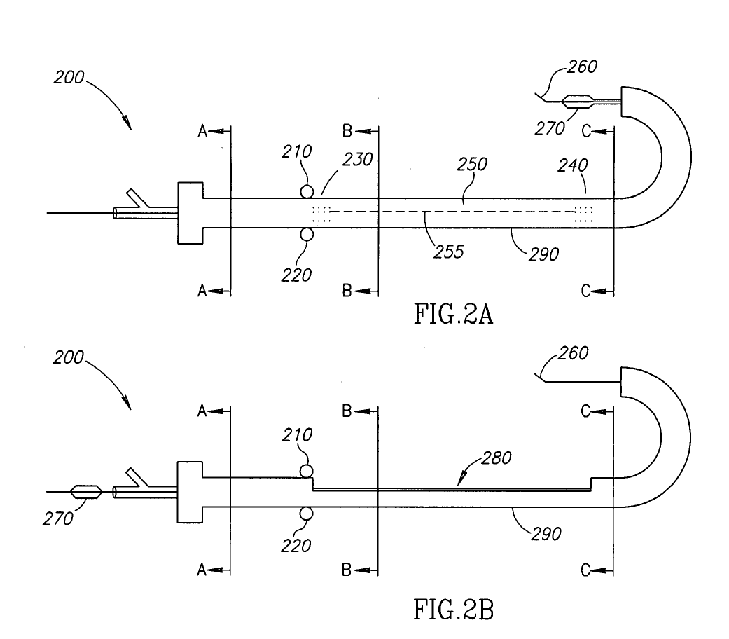

FIGS. 2A and 2B are side views of a catheter 200 according to an embodiment of the invention with an uncollapsed lumen and a collapsed lumen respectively.

FIG. 2A illustrates a catheter 200 with a lumen 250 and an un-collapsed wall 250 having sufficient cross-sectional area to accommodate insertion of guidewire 260 and balloon 270. Dotted line 255 indicates a center longitudinal axis of the lumen and will be employed as a reference point to illustrate reduction in cross-sectional area. Lines A-A; B-B and C-C indicate planes of cross-section which will be referred to hereinbelow. Catheter 200 is optionally equipped with flexible portions 230 and/or 240 which facilitate collapse of the lumen. In an exemplary embodiment of the invention, flexible portions 230 and/or 240 house and subsequently accommodate the collapse of structures used to reduce the volume of lumen 250. Flexible portions 230 and/or 240 are optionally stretchable areas. In an exemplary embodiment of the invention, stretching of portion 230 initiates collapse and collapsed structures are accommodated in portion 240. The collapse of lumen 250 may be controlled, for example, by one or more tensioning inputs 210 and/or 220.

Optionally, the cross-sectional area is adjustable over a significant portion of the length of the catheter, for example 10%, 25%, 35%, 45%, 55%, 65% 75%, 80%, 85% or 90% or lesser or greater or intermediate portions of the length of the catheter. In an exemplary embodiment of the invention, the cross-sectional area is adjustable over only a portion of the length of the catheter and not the whole length. Optionally the most distal 20 cm, optionally 10 cm, optionally 5 cm, optionally 1 cm (or greater or intermediate lengths) of the catheter lumen are not collapsible.

Optionally, the cross-sectional area of the catheter is adjustable by, for example, 25%, 35%, 45%, 55%, 65% 75% or lesser or greater or intermediate values. In an exemplary embodiment of the invention, a total volume of contrast material which may reside in the catheter lumen at any given moment is reduced by one fifth, optionally one quarter, optionally one third, optionally one half, or lesser or greater or intermediate values.

Optionally, changes in cross-sectional area cause a perceived change in flow resistance characteristics which corresponds to a 40%, optionally 50%, optionally 70% or lesser or greater or intermediate reduction in catheter circumference. In an exemplary embodiment of the invention shielding of a portion of said cross-sectional area from a flow of contrast material produces the perceived change in flow resistance characteristics. Although collapsible portion 280 is depicted as a single continuous length, two or more collapsible portions 280, with intervening non collapsible portions, may optionally be provided. Optionally, this permits provision of longer collapsible lengths and/or simplifies engineering of collapse mechanisms.

In an exemplary embodiment of the invention, catheter 200 is designed with a non-reinforced portion 230 in a proximal portion of the catheter. Optionally, this placement makes operation of tensioning inputs 210 and/or 220 easier for the operator.

In an exemplary embodiment of the invention, catheter 200 is designed with flexible portions 230 and 240 in a portion of the catheter. Optionally, this placement simplifies a manufacturing process and/or reduces deformation of catheter 200 when lumen 250 is collapsed.

FIG. 2B illustrates how collapse of lumen wall section 280, optionally to midline 255, reduces a cross-sectional area of a portion of lumen 250. In an exemplary embodiment of the invention, this contributes to a significant reduction in the volume of lumen 250. Optionally, collapsible lumen wall 280 approaches midline 255 and causes a reduction in the cross-section of lumen 250 which approaches 50%. In an exemplary embodiment of the invention, collapsible wall 280 is two thirds of the length of catheter 200 and collapse causes a reduction in lumen cross-section of 50%. According to this exemplary embodiment of the invention, a reduction in volume of lumen 250 by about one third is achieved. According to additional embodiments of the invention, greater or lesser reductions in volume are achieved.

In an exemplary embodiment of the invention, a coronary catheter with a total length of approximately 1 meter contains a collapsible portion with a length of 50 to 70 cm, optionally about 60 cm.

In an exemplary embodiment of the invention, a peripheral catheter with a total length of about 45 cm includes a collapsible portion with a length of 20 to 25 to 30 cm.

As an illustrative example, a 6 French angioplasty catheter with an overall length of 1 M and a total lumen volume of about 25.7 cc is considered. Assuming a 60 cm collapsible portion with a reduction in cross-sectional area of 50%, a savings of about 7.71 cc of contrast material can be achieved by employing the present invention. If multiple imaging procedures are performed, the savings would increase proportionally.

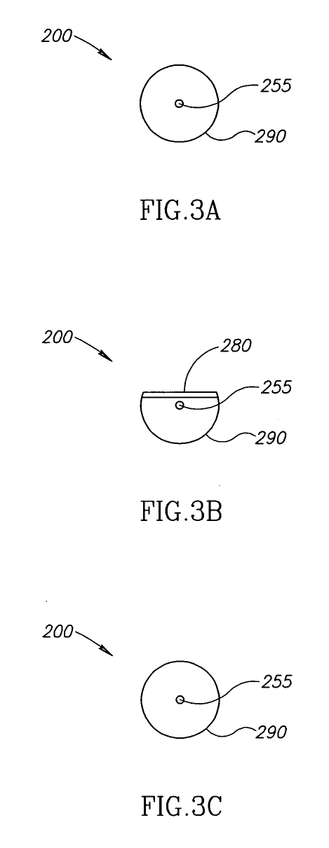

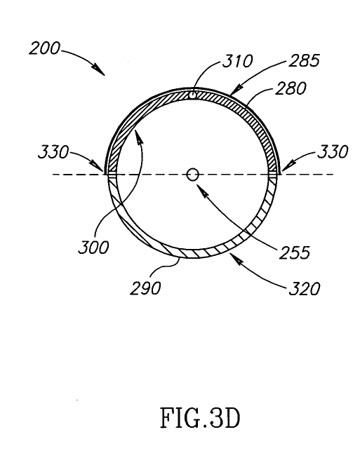

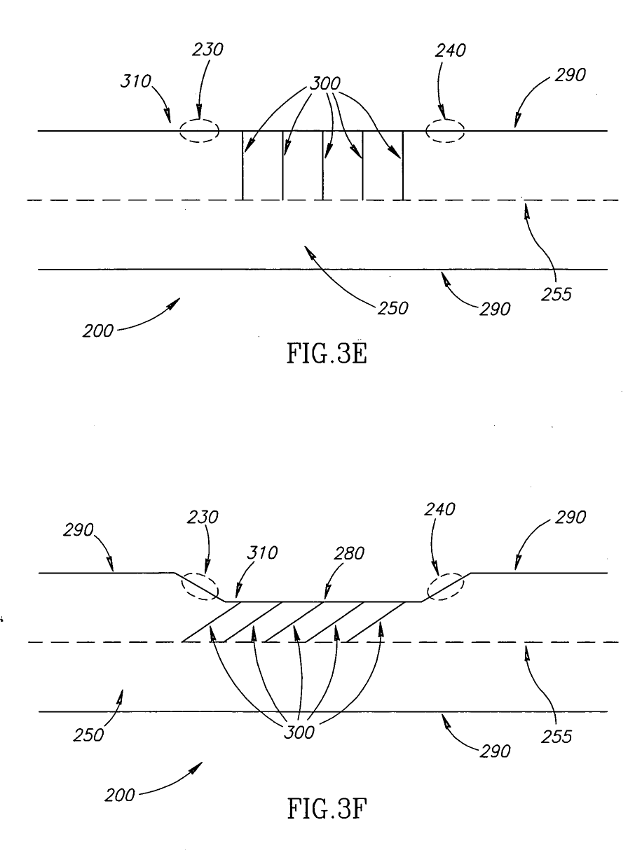

FIGS. 3A and 3C illustrate cross-sections of lumen 250 of catheter 200 at lines A-A and C-C respectively. At these points, no collapse occurs and the cross-sectional area of lumen 250 remains unchanged.

FIG. 3B illustrates a cross-section of lumen 250 of catheter 200 at line B-B while collapsed as shown in FIG. 2B. An exemplary method for achieving this collapse is set forth hereinbelow.

Exemplary Collapse Mechanism

According to one embodiment of the invention, collapse of the lumen wall is non-radial. According to alternate embodiments of the invention, collapse is radial.

In an exemplary embodiment of the invention, various functional elements of the collapse mechanism are embedded in a wall of lumen 250. One of ordinary skill in the art will be familiar with the practice of embedding structural supports in a catheter wall. Typically these structural supports are supplied as woven metal fibers embedded in a polymeric matrix. One of ordinary skill in the art will be capable of modifying the known embedding processes to incorporate relevant structural elements of a collapse mechanism into a wall of lumen 250. Optionally, an embedded collapse mechanism will leave the inner surface of lumen 250 and/or outer surface of catheter 200 smooth. In an exemplary embodiment of the invention, smoothness facilitates insertion of tools in lumen 250 and/or advance of catheter 200 along a guidewire and/or advance of catheter 200 along a blood vessel.

In an exemplary embodiment of the invention, portions (e.g. 230 and/or 240) of catheter 200 are supplied without reinforcement. Sections 230 and/or 240 are optionally flexible and/or expandable in a manner which allows an outer wall 290 to conform to changes imposed by collapse of wall section 280.

FIG. 3D is a cross-section illustration of operational components of an exemplary collapse mechanism to achieve the collapse depicted in FIGS. 2B and 3B. According to this exemplary collapse mechanism, a series of rigid arches 300 are deployed along the collapsible length 280 of catheter 200 between line A-A and C-C. Optionally, each arch 300 covers half of a circumference of lumen 250 so that a plane through a base of the arch describes a diameter (indicated by dotted line) of lumen 250. At least one tensioning member 310 connects arches 300.

Tensioning member 310 may be connected to each of arches 300 by any available connection method. Available connection methods include, but are not limited to, gluing, welding, tying and riveting. Operation of tensioning member 310 by tensioning input 210 and/or 220 causes tensioning member 310 to undergo axial translation approximately parallel to midline 255 (see arrow in FIG. 3E). This axial translation causes each arch to undergo a partial rotation with respect to pivot points 330. In an exemplary embodiment of the invention, a cover layer 285 (optionally an elastic layer) stretched between arches 300 is pulled towards centerline 255 as arches 300 rotate. In an exemplary embodiment of the invention, a lower half 320 of catheter wall 290 is constructed of a reinforced material. The reinforced material optionally provides resistance to deformation in lower half 320 as arched 300 exert force on pivot points 330. Optionally, the cover layer 285 is not elastic, but is flexible and attached to the arches so that the cover material is drawn down toward the center-line with the arches.

In an exemplary embodiment of the invention, clockwise rotation of a tensioning input 210 and/or 220 results in collapse of arches 300 and counterclockwise rotation results in restoration of the catheter to an uncollapsed state.

In an exemplary embodiment of the invention, at least two, optionally at least three, optionally five or more tensioning members 310 are connected to arches 300. Optionally, use of more than one tensioning member 310 prevents deformation of arches 300 during collapse. In an exemplary embodiment of the invention, each of tensioning inputs 210 and 220 is connected to one or more drive members 310 and operates the drive members in one direction to cause collapse and in a second direction to uncollapse lumen 250.

FIG. 3E is a side cross-sectional view of catheter 200 showing a series of arches 300 connected by a tensioning mechanism 310 in the form of a drive rod. Axial displacement (arrow) of drive rod 310 caused by a force applied through tensioning inputs 210 and/or 220 causes arches 300 to undergo rotational displacement with respect to pivot points 330.

FIG. 3F is a side view as in 3E illustrating the coordinated angular shift of arches 300. The coordinated angular shift brings collapsible wall section 280 towards midline 255 reducing a cross-sectional area of lumen 250. Sections 230 and/or 240 of outer wall 290 optionally stretch and/or extend to permit wall section 280 to move. Sections 230 and/or 240 may optionally be constructed of an elastomeric polymer and/or be accordion folded.

In an exemplary embodiment of the invention, tensioning inputs 210 and 220 are each adapted to cause translational motion of a drive shaft 310 in an opposite direction. According to this embodiment, operation of input 210 causes collapse of wall section 280 and operation of input 220 restores catheter 200 to an uncollapsed state.

In an additional exemplary embodiment of the invention, a single input mechanism operates against a constant resistive force. The constant resistive force may be supplied, for example by an inherent elasticity of drive mechanism 310 and/or by a spring or other elastic component connected to a distal portion of drive mechanism 310.

After contrast material injection is complete, drive mechanism 310 is subject to an opposite axial motion, arches 300 return to the upright position of FIG. 3E and catheter 200 has a lumen 250 with its full volume as depicted in FIG. 2A. At this point, balloon 270 or a stent may be advanced along guidewire 260 to a distal portion of the catheter as illustrated in FIG. 2A.

In an exemplary embodiment of the invention, wall 290 includes one or more expandable portions which expand inwards towards midline 255. Expansion may optionally be achieved by inflation. Optionally, expandable portions are supplied along a partial length of the catheter, for example from line A-A to C-C as indicated in FIG. 2. The expandable portions expand to reduce a cross-section of lumen 250 without altering external appearance of the catheter.

In an exemplary embodiment of the invention, a liquid displacement member is introduced into lumen 250 to reduce a cross-sectional area of lumen 250 which can be occupied by contrast material. Optionally, the liquid displacement member has a constant diameter. Optionally, the liquid displacement member is provided as an inflatable balloon.

These collapse mechanism are presented as examples only and do not necessarily limit the scope of the invention. Other collapse mechanisms may allow for a greater or lesser amount of collapse.

Alteration of Handling Characteristics

In an exemplary embodiment of the invention, handling characteristics, as perceived by an operator of the invention are altered by narrowing at least a portion of the flow path of the contrast material. Optionally, narrowing at least a portion of the flow path is achieved by altering a cross-sectional area of a significant portion of the lumen as described above. In an exemplary embodiment of the invention, this contributes to an additional savings by causing a reduction in the second aliquot as described above.

In an exemplary embodiment of the invention, narrowing at least a portion of the flow path is achieved by imposing a narrow aperture obstruction in the flow path. Optionally, the narrow aperture obstruction is imposed outside and/or inside a lumen of the catheter.

In an exemplary embodiment of the invention, the catheter is equipped with a section outside the body characterized by a high degree of tensile memory. The high degree of tensile memory permits an operator to pinch, optionally with a tool, the section to increase a resistance to flow.

In an exemplary embodiment of the invention, the flow path of the contrast material includes a needle, optionally 18g (gauge), optionally 19g, optionally 21g, optionally 23g, optionally 25g, optionally 27g or lesser or greater or intermediate values. Optionally, the needle is 0.5 inches long, optionally 1 inch, optionally 1.5 inches or lesser or greater or intermediate values. The needle increases a resistance to flow.

In an exemplary embodiment of the invention, the operator responds to increased flow resistance by increasing injection pressure.

Potential Benefits of Some Embodiments the Invention

In an exemplary embodiment of the invention, use of catheters equipped with a collapse mechanism according to the invention permits a dramatic reduction in the amount of contrast material required for a medical imaging procedure. In an exemplary embodiment of the invention, the volume reduction approaches 50%, optionally 75%. The degree of volume reduction achieved by any specific embodiment of the invention may depend upon the length of the catheter involved in the volume reduction.

In an exemplary embodiment of the invention, a portion of the reduction is in the first aliquot of contrast material actively injected for imaging. Reduction in the size of the first aliquot is optionally due to an operator-perceived change in handling characteristics of the catheter. In an exemplary embodiment of the invention, an operator perceives a wide bore catheter as having a narrower bore because increased resistance to injection is encountered. Optionally, the operator compensates by applying increased injection force.

In an exemplary embodiment of the invention, a portion of the reduction is in the second aliquot of contrast material which remains in the catheter after injection is completed. Reduction in the size of the second aliquot is optionally due to an increase in a cross-sectional area of the lumen after imaging is complete so that the lumen is only partially filled with contrast material. Optionally, a medical tool advancing through the partially filled lumen pushes less contrast material through the lumen than a same tool advancing through a lumen completely filled with contrast material.

This reduction will, in turn, reduce the need for toxicity intervention which is typically based upon intravenous infusion. This is expected to contribute to a reduced need for monitoring by medical personnel and/or a reduced labor input by medical personnel to administer infusions.

Theoretical explanations of the operative principle of devices and/or methods according to the present invention should not be construed as necessarily limiting the invention, but may be used for design decisions in some embodiments.

In the description and claims of the present application, each of the verbs “comprise”, “include” and “have” as well as any conjugates thereof, are used to indicate that the object or objects of the verb are not necessarily a complete listing of members, components, elements or parts of the subject or subjects of the verb.

The present invention has been described using detailed descriptions of embodiments thereof that are provided by way of example and are not intended to necessarily limit the scope of the invention. In particular, numerical values may be higher or lower than ranges of numbers set forth above and still be within the scope of the invention. The described embodiments comprise different features, not all of which are required in all embodiments of the invention. Some embodiments of the invention utilize only some of the features or possible combinations of the features. Alternatively or additionally, portions of the invention described/depicted as a single unit may reside in two or more separate physical entities which act in concert to perform the described/depicted fiction. Alternatively or additionally, portions of the invention described/depicted as two or more separate physical entities may be integrated into a single physical entity to perform the described/depicted function. Variations of embodiments of the present invention that are described and embodiments of the present invention comprising different combinations of features noted in the described embodiments can be combined in all possible combinations including, but not limited to use of features described in the context of one embodiment in the context of any other embodiment. The scope of the invention is limited only by the following claims.

All publications and/or patents and/or product descriptions cited in this document are fully incorporated herein by reference to the same extent as if each had been individually incorporated herein by reference.

1. A method of reducing an amount of contrast material delivered to a patient during an imaging procedure, the method comprising:
(a) injecting contrast material through a catheter with a first cross-section; and
(b) increasing said cross-section of said lumen to a second cross-section greater than said first cross-section along a portion of the lumen comprising 10% to 90% of a length of said lumen;
wherein said increasing occurs after said injection.

2. A method according to claim 1, wherein said increasing said cross-section of said lumen is applied along a portion of the lumen comprising 15% to 80% of a length of said lumen.

3. A method according to claim 2, wherein said increasing said cross-section of said lumen is applied along a portion of the lumen comprising 25% to 75% of a length of said lumen.

4. A method according to claim 3, wherein said increasing said cross-section of said lumen is applied along a portion of the lumen comprising 40% to 70% of a length of said lumen.

5. A method according to claim 4, wherein said increasing said cross-section of said lumen is applied along a portion of the lumen comprising 50% to 70% of a length of said lumen.

6. A method according to claim 1, wherein said increasing said cross-section of said lumen includes removing an object from said lumen.

7. A method according to claim 1, additionally comprising:
(c) providing a catheter with said second cross-section; and
(d) reducing said second cross-section to said first cross-section; and
(e) maintaining said first cross-section during cessation of said injecting.

8. A method according to claim 7, wherein said reducing occurs during said injection.

9. A method according to claim 1, additionally comprising conducting an imaging procedure after or during (a).

10. A method according to claim 1, additionally comprising inserting a medical tool via said lumen of said catheter after (b).

11. A method according to claim 10, additionally comprising performing a procedure with said tool.

12. A coronary catheter, the catheter comprising;
(a) a lumen having a cross-sectional area; and
(b) a collapse mechanism adapted to switch said cross-sectional area between a first configuration and a second configuration over a portion of the catheter, said portion comprising between 10% and 90% of a total catheter length.

13. A catheter according to claim 12, wherein said portion of the catheter is a contiguous portion.

14. A catheter according to claim 12, wherein said portion of the catheter is a non-contiguous portion.

15. A catheter according to claim 12, wherein said portion of the catheter does not include a distal region of the catheter.

16. A catheter according to claim 15, wherein said distal region of the catheter has a length in the range of 1 to 20 cm measured from a distal tip of the catheter.

17. A catheter according to claim 12, wherein said collapse mechanism provides a non-radial change in said cross-sectional area.

18. A catheter according to claim 12, wherein said collapse mechanism is capable of reducing said cross-sectional area by at least 25%.

19. A catheter according to claim 12, wherein said collapse mechanism is capable of reducing said cross-sectional area by no more than 50%.

20. A catheter according to claim 12, wherein said switch between said first configuration and said second configuration alters a total volume of said lumen of the catheter by at least 33%.

21. A catheter according to claim 12, supplied as an arterial catheter.

22. A catheter according to claim 12, characterized in that it has sufficient pushability to permit insertion in the face of a resistive force.

23. A catheter according to claim 12, comprising a lumen which is partially, but not completely, filled with a contrast material.

24. A catheter according to claim 12, wherein said collapse mechanism comprises:
(a) a series of structures deployed along a length of a wall of a lumen of a catheter, each of said structures attached to said wall; and
(b) a mechanism adapted to move said structures in concert to alter said cross-sectional area of said lumen.

25. A catheter according to claim 24, wherein said series includes at least two sub-series and a length of wall of said lumen devoid of said structures is interposed between each pair of said at least two sub-series.

26. A catheter according to claim 24, wherein said structures include arches.

27. A catheter according to claim 24, wherein said structures are attached to said wall by being embedded therein.

28. A catheter according to claim 24, wherein said structures are characterized by a height which does not exceed a radius of said lumen.

29. A catheter according to claim 24, wherein said structures are characterized by a height equal to a radius of said lumen.

30. A mechanism for adjusting a cross-sectional area of a lumen of a catheter, the mechanism comprising:
(a) a series of structures deployed along a length of a wall of a lumen of a catheter; and
(b) a mechanism adapted to rotate said structures in concert to alter a cross-sectional area of said lumen.

31. A method of reducing an amount of contrast material delivered to a patient during an imaging procedure, the method comprising:
(a) reducing a cross-sectional area of a portion of a lumen of a catheter from an original cross-sectional area to a reduced cross-sectional area;
(b) injecting contrast material; and
(c) increasing said cross-sectional area of said lumen to a cross-sectional area greater than said reduced cross-sectional area.

32. A method according to claim 31, wherein said increasing comprises restoration of said cross-sectional area of said lumen at least to said original cross-sectional area.

33. A catheter with an adjustable lumen volume, the catheter comprising a mechanism to reduce a cross-sectional area of at least two separate portions of the catheter, said separate portions characterized by an aggregate length comprising between 10% and 90% of a total catheter length.

34. A catheter according to claim 33 wherein said mechanism does not reduce a cross-sectional area of at least one intervening portion between said two separate portions.

35. A method of reducing an amount of contrast material injected during an imaging procedure conducted with a dual purpose catheter, the method comprising causing an increased resistance to flow of a contrast material.

36. A method according to claim 35, wherein said increased resistance to flow causes an increase in injection pressure.

37. A method according to claim 35, wherein said increased resistance to flow alters flow properties of said contrast material during ejection from a catheter.

38. A system for reducing an amount of contrast material injected during an imaging procedure conducted with a dual purpose catheter, the system comprising:
(a) a dual purpose catheter operably connectable to a manual injector;
(b) said manual injector containing a contrast material; and
(c) a regulation mechanism adapted to increase flow resistance to a degree perceivable by an operator of said manual injector.

39. A system according to claim 38, wherein said regulation mechanism operates by altering a cross-sectional area of a portion of a lumen of said catheter.

40. A catheter with an adjustable fluid resistance characteristic, the catheter comprising:
(a) a lumen with a cross-sectional area; and
(b) a shielding mechanism to shield a portion of said cross-sectional area from a flow of contrast material over 10 to 90% of a length of said lumen.

41. A catheter according to claim 40, wherein operation of said mechanism shields at least 40% of said cross-sectional area from said flow of contrast material.

42. A catheter according to claim 41, wherein operation of said mechanism shields at least 50% of said cross-sectional area from said flow of contrast material.

43. A catheter according to claim 42, wherein operation of said mechanism shields at least 70% of said cross-sectional area from said flow of contrast material.

44. A catheter, said catheter comprising a lumen, said lumen characterized by a cross-sectional area sufficient to permit passage of a tool and contrast material occupying no more than 50% of said cross-sectional area of said lumen over at least 50% of a length of said lumen.
